(19) United States
(12) Patent Application Publication
(10) Pub. No.: US 2010/0069814 A1
(43) Pub. Date: Mar. 18, 2010

(54) SYSTEM AND METHOD FOR UTILIZING MICROBUBBLES AND LIPOSOMES AS VIRAL SEQUESTERING AGENTS

(76) Inventors: Anthony V. Borgia, Las Vegas, NV (US); John Perry, Las Vegas, NV (US)

Correspondence Address: GREENBERG TRAURIG (LV), 3773 HOWARD HUGHES PARKWAY, Suite 400 North, LAS VEGAS, NV, 89169, US

(21) Appl. No.: 12/211,748

(22) Filed: Sep. 16, 2008

(51) Int. Cl. A61M 37/00; A61K 9/12; A61K 9/127

(52) U.S. Cl. 604/5.02; 424/45; 424/450

(57) ABSTRACT

A system and method of utilizing microbubbles and liposomes as viral sequestering agents. Microbubbles and liposomes having polyanions and conjugation systems are injected into the bloodstream of a subject human or animal. The microbubbles and/or liposomes attract virus particles including envelope viruses. After the microbubbles and/or liposomes have circulated in the blood for a pre-established time period, the microbubbles and/or liposomes along with attached virus particles are separated from the blood of the subject by passing the blood over or through a substrate or using a centrifuge. The substrate includes a conjugation system which has an affinity for the polyanions of the microbubbles and/or liposomes. As the microbubbles and/or liposomes and attached virus particles are removed from the blood, the viral titer of the subject is lowered. The microbubbles and/or liposomes may also be mixed with blood and filtered outside of the subject such that the microbubbles and/or liposomes are never in the bloodstream of the subject.

### FIELD OF THE INVENTION

The embodiments of the present invention relate to microbubbles and their use as sequestering agents to lower viral titer levels.

### BACKGROUND

Microbubbles are very small gas bubbles commonly used as contrast agents in ultrasonography. In such applications, the microbubbles are injected intravenously into a subject's bloodstream and detonated at a desired location using ultrasound waves. Conventionally, microbubbles are fabricated of a protein, such as albumin, and are filled with an inert gas, such as perfluoropropane. While microbubbles can take on various sizes, microbubbles are commonly in the magnitude of 3 microns in diameter and number 1.6×10⁹ per milliliter of solution. Liposomes are similar to microbubbles but contain no gas.

Bacterial infections are routinely treated using antibiotics, but viral infections have limited treatment options. Indeed, in many instances the body's immune system is left to defeat the viral infection over time. However, allowing ‘nature to take it course’ is not a comforting treatment protocol for the ill or sick subject.

Although various uses (e.g., contrast, oxygenator, therapeutic agents, etc.) for microbubbles and liposomes have been devised, it would be advantageous to utilize microbubbles and liposomes as viral sequestering agents to assist the body's immune system to more quickly and readily defeat viral infections.

### SUMMARY

Accordingly, a first embodiment of the present invention is a microbubble comprising: a gas; a polyanion; a conjugation system; and one or more of the following: a protein; a lipid; a cationic protein and a cationic lipid. Another embodiment is a liposome comprising: a polyanion; a conjugation system; and one or more of the following: a protein; a lipid; a cationic protein and a cationic lipid. In one embodiment, the polyanion is a phosphorothioate DNA oligonucleotide and the conjugation system is biotin. Set forth in the detailed description below are exemplary lists of possible lipids, gases, polyanions and conjugation systems which are suitable for the embodiments of the present invention.

One method embodiment of the present invention comprises: injecting a subject with microbubbles having a cationic protein; a gas; at least one polyanion; and a conjugation system; allowing said microbubbles to remain in said subject for a pre-established time period; and once said pre-established time period has expired, passing said subject's blood through a substrate configured to remove said microbubbles and attached virus particles. A second method comprises: selectively extracting a subject's blood; passing said blood through a solution of microbubbles having a cationic protein; a gas; at least one polyanion; and a conjugation system; after a pre-established time period, passing said blood and microbubbles through a substrate configured to remove said microbubbles and attached virus particles; and re-introducing filtered blood back into said subject. These methods work with liposomes as well.

One system embodiment of the present invention for lowering a viral titer comprises: microbubbles having a cationic protein; a gas; at least one polyanion; and a conjugation system; and a substrate including a conjugation system.

With the embodiments of the present invention, viruses are removed from the blood of a human or animal thereby lowering the subject's viral titer such that the immune system of the subject is better able to defeat the virus in a timely fashion.

Other variations, embodiments and features of the present invention will become evident from the following detailed description, drawings and claims.

### BRIEF DESCRIPTION OF THE DRAWINGS

FIG. 1 illustrates an exemplary naturally occurring DNA;

FIG. 2 illustrates an exemplary phosphorothioate DNA oligonucleotide;

FIGS. 3a and 3b illustrate exemplary phosphorothioate RNA oligonucleotides;

FIG. 4 illustrates a microbubble having phosphorothioate DNA oligonucleotides joined thereto;

FIG. 5 illustrates a series of the microbubbles shown in FIG. 4 after injection into a bloodstream;

FIG. 6 illustrates a series of the microbubbles with virus particles attached thereto passing by a substrate; and

FIGS. 7 and 8 illustrate flow charts detailing two methods according to the embodiments of the present invention.

### DETAILED DESCRIPTION

For the purposes of promoting an understanding of the principles in accordance with the embodiments of the present invention, reference will now be made to the embodiments illustrated in the drawings and specific language will be used to describe the same. It will nevertheless be understood that no limitation of the scope of the invention is thereby intended. Any alterations and further modifications of the inventive feature illustrated herein, and any additional applications of the principles of the invention as illustrated herein, which would normally occur to one skilled in the relevant art and having possession of this disclosure, are to be considered within the scope of the invention claimed.

A virus is a sub-microscopic infectious agent that is unable to grow or reproduce outside a host cell. Each viral particle (“virion”) consists of genetic material, DNA or RNA, within a protective protein coat called a capsid. The capsid shape varies from simple helical forms to more complex structures with tails or an envelope. Treatment of viral infections is often limited to the body's immune system. In other situations, vaccines are used to provide resistance to infection while antiviral drugs are used to treat symptoms of viral infections. The embodiments of the present invention seek to reduce the number of viruses in the bloodstream thereby allowing the body's immune system a better opportunity to eradicate the remaining viruses.

There is set out below a list composed of lipids that may be used to form the microbubbles described herein. The list is not exhaustive since it is possible to use other lipids. Exemplary lipids for use in the embodiments of the present invention include: fatty acids, lysolipids, phosphatidylcholine with both saturated and unsaturated lipids including dioleoylphosphatidylcholine; dimyristoylphosphatidylcholine; dipentadecanoylphosphatidylcholine; dilauroylphosphatidylcholine; dipalmitoylphosphatidylcholine (DPPC); distearoylphosphatidylcholine (DSPC); phosphatidylethanolamines such as dioleoylphosphatidylethanolamine and dipalmitoylphosphatidylethanolamine (DPPE); phosphatidylserine; phosphatidylglycerol; phosphatidylinositol; sphingolipids such as sphingomyelin; glycolipids such as ganglioside GM1 and GM2; glucolipids; sulfatides; glycosphingolipids; phosphatidic acids such as dipalymitoylphosphatidic acid (DPPA); palmitic acid; stearic acid; arachidonic acid; oleic acid; lipids bearing polymers such as polyethyleneglycol, i.e., PEGylated lipids, chitin, hyaluronic acid or polyvinylpyrrolidone; lipids bearing sulfonated mono-, di-, oligo- or polysaccharides; cholesterol, cholesterol sulfate and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylamine; cardiolipin; phospholipids with short chain fatty acids of 6-8 carbons in length; synthetic phospholipids with asymmetric acyl chains (e.g., with one acyl chain of 6 carbons and another acyl chain of 12 carbons); ceramides; non-ionic liposomes including niosomes such as polyoxyethylene fatty acid esters, polyoxyethylene fatty alcohols, polyoxyethylene fatty alcohol ethers, polyoxyethylated sorbitan fatty acid esters, glycerol polyethylene glycol oxystearate, glycerol polyethylene glycol ricinoleate, ethoxylated soybean sterols, ethoxylated castor oil, polyoxyethylene-polyoxypropylene polymers, and polyoxyethylene fatty acid stearates; sterol aliphatic acid esters including cholesterol sulfate, cholesterol butyrate, cholesterol iso-butyrate, cholesterol palmitate, cholesterol stearate, lanosterol acetate, ergosterol palmitate, and phytosterol n-butyrate; sterol esters of sugar acids including cholesterol glucuroneide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid, accharic acid, and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; longchain alcohols including n-decyl alcohol, lauryl alcohol, myristyl alcohol, cetyl alcohol, and n-octadecyl alcohol; 6-(5-cholesten-3.beta.-yloxy)-1-thio-.beta.-D-galactopyranoside; digalactosyldiglyceride; 6-(5-cholesten-3.beta.-yloxy)hexyl-6-amino-6-deoxy-1-thio-.beta.-D-galactopyranoside; 6-(5-cholesten-3.beta.-yloxy)hexyl-6-amino-6-deoxyl-1-thio-.alpha.-D-mannopyranoside; 12-(((7′-diethylaminocoumarin-3-yl)carbonyl)methylamino)-octadecanoic acid; N-[12-(((7′-diethylaminocoumarin-3-yl)carbonyl)methyl-amino)octadecanoyl]-2-aminopalmitic acid; cholesteryl)4′-trimethylammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoyl-glycerophosphoe thanolamine and palmitoylhomocysteine, and/or combinations thereof.

There is set out below a list composed of potential gases that may be used to form the microbubbles described herein. The list is not exhaustive since it is possible to use other gases. Exemplary gases for use in the embodiments of the present invention include: hexafluoro acetone, isopropyl acetylene, allene, tetrafluoro-allene, boron trifluoride, isobutane, 1,2-butadiene, 2,3-butadiene, 1,3-butadiene, 1,2,3-trichloro-2-fluoro-1,3-butadiene, 2-methyl-1,3-butadiene, hexafluoro-1,3-butadiene, butadiyne, 1-fluoro-butane, 2-methyl-butane, decafluorobutane, 1-butene, 2-butene, 2-methyl-1-butene, 3-methyl-1-butene, perfluoro-1-butene, perfluoro-2-butene, 4-phenyl-3-butene-2-one, 2-methyl-1-butene-3-yne, butyl nitrate, 1-butyne, 2-butyne, 2-chloro-1,1,1,4,4,4-hexafluorobutyne, 3-methyl-1-butyne, perfluoro-2-butyne, 2-bromobutyraldehyde, carbonyl sulfide, crotononitrile, cyclobutane, methyl-cyclobutane, octafluoro-cyclobutane, perfluorocyclobutene, 3-chlorocyclopentene, octafluorocyclopentene, cyclopropane, 1,2-dimethyl-cyclopropane, 1,1-dimethylcyclopropane, 1,2-dimethyl-cyclopropane, ethylcyclopropane, methylcyclopropane, diacetylene, 3-ethyl-3-methyl diaziridine, 1,1,1-trifluorodiazoethane, dimethylamine, hexafluorodimethylamine, dimethylethylamine, bis(dimethylphosphine)amine, perfluorohexane, 2,3-dimethyl-2-norbornane, perfluorodimethylamine, dimethyloxonium chloride, 1,3-dioxolane-2-one, 4-methyl-1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1,1-dichloro-2-fluoroethane, 1-chloro-1,1,2,2-tetrafluoroethane, 2-chloro-1,1-difluoroethane, chloroethane, chloropentafluoroethane, dichlorotrifluoroethane, fluoroethane, hexafluoroethane, nitropentafluoroethane, nitrosopentafluoroethane, perfluoroethylamine, ethyl vinyl ether, 1,1-dichloroethane, 1,1-dichloro-1,2-difluoroethane, 1,2-difluoroethane, methane, trifluoromethanesulfonylchloride, trifluoromethanesulfonylfluoride, bromodifluoronitrosomethane, bromofluoromethane, bromochlorofluoromethane, bromotrifluoromethane, chlorodifluoronitromethane, chlorodinitromethane, chlorofluoromethane, chlorotrifluoromethane, chlorodifluoromethane, dibromodifluoromethane, dichlorodifluoromethane, dichlorofluoromethane, difluoromethane, difluoroiodomethane, disilanomethane, fluoromethane, iodomethane, iodotrifluoromethane, nitrotrifluoromethane, nitrosotrifluoromethane, tetrafluoromethane, trichlorofluoromethane, trifluoromethane, 2-methylbutane, methyl ether, methyl isopropyl ether, methyllactate, methylnitrite, methylsulfide, methyl vinyl ether, neon, neopentane, nitrogen (N.sub.2), nitrous oxide, 1,2,3-nonadecane-tricarboxylic acid-2-hydroxytrimethylester, 1-nonene-3-yne, oxygen (O.sub.2), 1,4-pentadiene, n-pentane, perfluoropentane, 4-amino-4-methylpentan-2-one, 1-pentene, 2-pentene(cis), 2-pentene(trans), 3-bromopent-1-ene, perfluoropent-1-ene, tetrachlorophthalic acid, 2,3,6-trimethylpiperidine, propane, 1,1,1,2,2,3-hexafluoropropane, 1,2-epoxypropane, 2,2-difluoropropane, 2-aminopropane, 2-chloropropane, heptafluoro-1-nitropropane, heptafluoro-1-nitrosopropane, perfluoropropane, propene, hexafluoropropane, 1,1,1,2,3,3-hexafluoro-2,3dichloropropane, 1-chloropropane, chloropropane-(trans), 2-chloropropane, 3-fluoropropane, propyne, 3,3,3-trifluoropropyne, 3-fluorostyrene, sulfur hexafluoride, sulfur (di)-decafluoride(S.sub.2F.sub.10), 2,4-diaminotoluene, trifluoroacetonitrile, trifluoromethyl peroxide, trifluoromethyl sulfide, tungsten hexafluoride, vinyl acetylene, vinyl ether, and xenon.

There is set out below a list composed of potential cationic lipids that may be used to form the microbubbles described herein. The list is not exhaustive since it is possible to use other cationic lipids. Exemplary cationic lipids for use in the embodiments of the present invention include: distearoyl phosphatidylcholine, 1,2-distearoyl-3-trimetylammoniumpropane, 1,2-Dimyristoyl-3-Trimethylammonium-Propane, 1,2-Dipalmitoyl-3-Trimethylammonium-Propane, 1,2-Dioleoyl-3-Trimethylammonium-Propane, 1,2-Dimyristoyl-3-Dimethylammonium-Propane, 1,2-Dipalmitoyl-3-Dimethylammonium-Propane, 1,2-Distearoyl-3-Dimethylammonium-Propane, 1,2-Dioleoyl-3-Dimethylammonium-Propane, 3β-[N—(N′,N′-Dimethylaminoethane)-carbamoyl]Cholesterol Hydrochloride, Dimethyldioctadecylammonium, 1,2-Dilauroyl-sn-Glycero-3-Ethylphosphocholine, 1,2-Dimyristoyl-sn-Glycero-3-Ethylphosphocholine, 1,2-Dipalmitoyl-sn-Glycero-3-Ethylphosphocholine, 1,2-Distearoyl-sn-Glycero-3-Ethylphosphocholine, 1,2-Dioleoyl-sn-Glycero-3-Ethylphosphocholine, 1-Palmitoyl-2-Oleoyl-sn-Glycero-3-Ethylphosphocholine, sterols and all cationic lipids.

In one embodiment, phosphorothioate DNA oligonucleotides are used in combination with microbubbles to achieve the objectives of the present invention. Phosphorothioate DNA oligonucleotides are modified analogs of naturally occurring DNA. Phosphorothioate DNA oligonucleotides are amphipathic having both hydrophobic and hydrophilic properties. Importantly, phosphorothioate DNA oligonucleotides are able to link or bond to glycoproteins present on the surface of an enveloped virus. One exemplary glycoprotein is the HIV gp120 envelope glycoprotein. Many other glycoproteins exist and are relevant to the embodiments of the present invention including but not limited to phosphorothioate 2′O-methyl DNA and RNA polycystosine oligonucleotides and degenerate phosphorothioate randomer oligonucleotides, polyoxometalates, polysulfonates, polyhydroxycarboxylates and sulfonated saccharides.

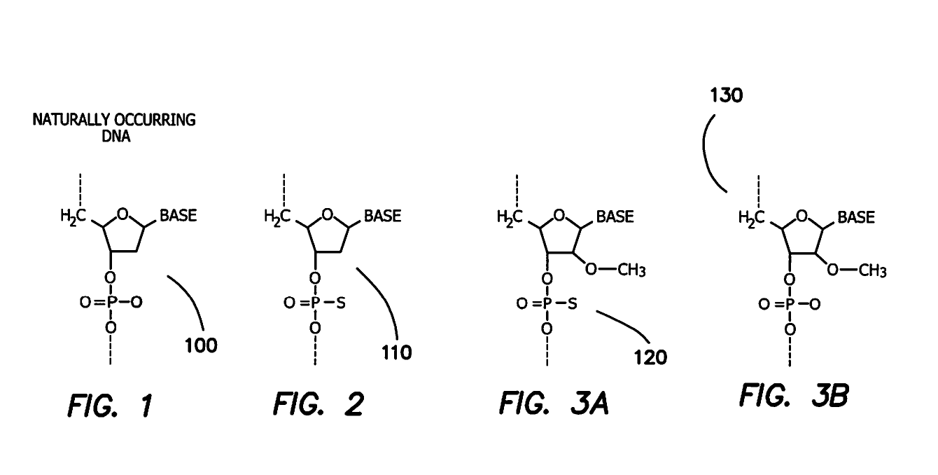

FIG. 1 shows naturally occurring DNA article generally referred to by reference numeral 100. The DNA article 100 is polyanionic and highly unstable. FIG. 2 shows an exemplary phosphorothioate DNA oligonucleotide (randomer 1) 110 of the type that may be used with the embodiments of the present invention. Phosphorothioate DNA oligonucleotide (randomer 1) 110 is polyanionic, hydrophobic and stable in vivo. FIGS. 3a and 3b show phosphorothioate 2′-O-methyl RNA (randomer 2) 120 and phosphorothioate 2′-O-methyl RNA (randomer 3) 130, respectively. Phosphorothioate 2′-O-methyl RNA (randomer 2) 120 is polyanionic, hydrophobic and stable in vivo and phosphorothioate 2′-O-methyl RNA (randomer 3) 130 is polyanionic and stable in vivo. Exemplary polyanions for use in the embodiments of the present invention include: phosphorothioate 2′O-methyl DNA and RNA polycystosine oligonucleotides and degenerate phosphorothioate randomer oligonucleotides, polyoxometalates, polysulfonates, polyhydroxycarboxylates and sulfonated saccharides.

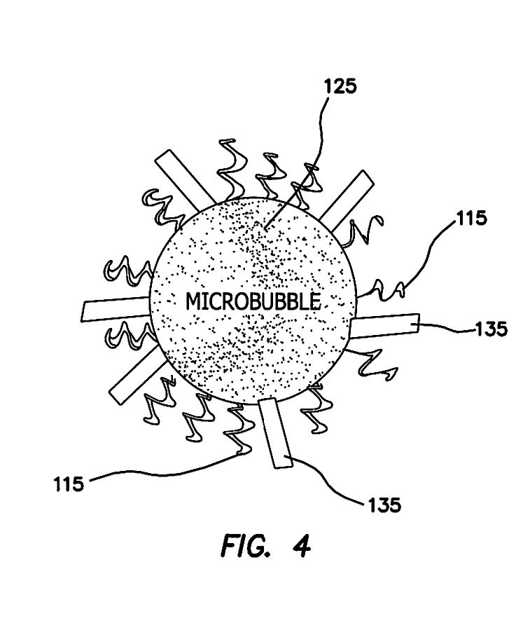

In one embodiment of the present invention, as shown in FIG. 4, phosphorothioate DNA oligonucleotides 115 and/or phosphorothioate RNA oligonucleotides are formed with, or attached to, microbubbles 125. The microbubble 125 is cationic (i.e., positively charged). In one embodiment, PEG-Biotin 135 (conjugation system) may is formed with, or attached to, the microbubble 125. The phosphorothioate DNA oligonucleotides 115 and/or phosphorothioate RNA oligonucleotides and PEG-Biotin 135. In another embodiment, phosphorothioate DNA oligonucleotides 115 and/or phosphorothioate RNA oligonucleotides and PEG-Biotin are formed with the shell of the microbubbles 125. Exemplary conjugation systems for use in the embodiments of the present invention include: Succinimidyl 6-hydrazinonicotinamide acetone hydrazone, Succinimidyl 4-formylbenzoate, all Hy Nic conjugation systems, all His-tag (polyhistidine) protein conjugation systems, streptavidin, avidin biotinylated polyethylene glycol, biotinylated proteins, biotinylated olinucleotides and all biotinylated conjugation systems, all affinity conjugation.

In one embodiment, the microbubbles include a PEG-biotin incorporated into the shell or surface of the microbubble, along with a Streptavidin linker and a target attachment protein that has been biotinylated. Once formed, the microbubbles are incubated with the appropriate phosphorothioate DNA oligonucleotides to form the complete microbubbles for injection into a patient. In this embodiment, the PEG-Biotin or other polyanion has an affinity for the target attachment protein.

In one embodiment, the components of the microbubble (e.g., protein, lipid, cationic protein or lipid, conjugation system, etc.) are formed in the shell of the microbubble. In one embodiment, the polyanion is attached to the microbubbles after an incubation period. Alternatively, the polyanion may be formed with the microbubble shell.

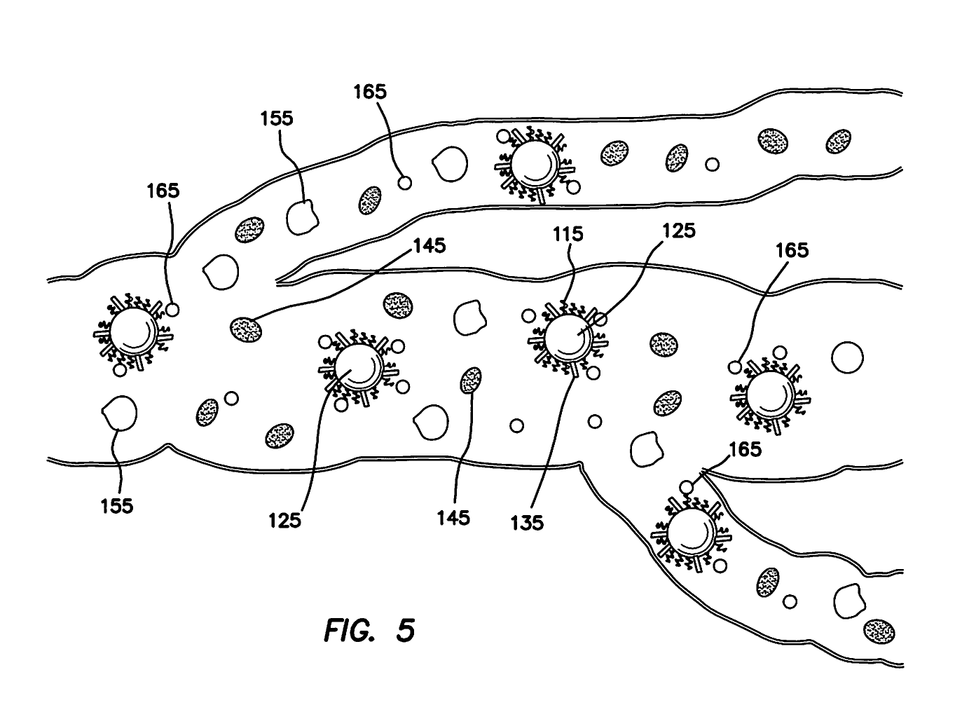

In one embodiment, the microbubbles 125, as shown in FIG. 4, are injected into the bloodstream of a subject person (or animal). The microbubbles 125 circulate through the bloodstream of the subject as shown in FIG. 5. While circulating in the bloodstream, the microbubbles 125 are intermixed with red blood cells 145, white blood cells 155 and certain viruses 165. The negatively charged phosphorothioate DNA oligonucleotides 115 and/or phosphorothioate RNA oligonucleotides (or other polyanions) attract the envelope viruses 165 since viral proteins are amphipathic as are many polyanions such the two have an affinity for each other. As the microbubbles 125 circulate through the bloodstream, as shown in FIG. 5, the microbubbles 125 attract and bond with the envelope viruses 165. As is well known, Streptavidin has a strong affinity for biotin including PEG-Biotin 135 (or other conjugation systems). After a pre-established time period, the microbubbles 125 and attached envelope viruses 165 are removed from the bloodstream as set forth below. In one embodiment, the microbubbles 125 remain in the bloodstream for approximately 2 hours. However, the time may be less or more and as dictated by the lifespan of the microbubble 125. Envelope viruses 165 which may be sequestered using the embodiments of the present invention are set forth in the following six tables designated Hepatitis Viruses, Respiratory Viruses, Immunodeficiency Viruses, Herpes Viruses, Biodefense Viruses and Emerging and Tropical Viruses. It will be recognized by those skilled in the art that many other viruses may be targeted as well.

TABLE 1
Hepatitis Viruses

Hepatitis C
Duck HBV
Hepatitis B (HBV)

TABLE 2
Respiratory Viruses

Influenza A
Influenza B
Human metapneumovirus
Respiratory syncytial (RSV)

TABLE 3
Immunodeficiency Viruses

Friend leukemia
HIV-1

TABLE 4
Herpes Viruses

Cytomegalovirus (CMV)
Varicella zoster (VZV)
Herpes simplex 1 (HSV-1)
Herpes simplex 2 (HSV-2)
HHV-6A and 6B
Epstein-Barr (EBV) Human herpes

TABLE 5
Biodefense Viruses

Lassa fever
Vaccinia (smallpox surrogate)
Mousepox (ectomella)
Ebola
Marbug

TABLE 6
Emerging & Tropical Viruses

Hantavirus
Venezuelan equine encephalitis
Tick born encephalitis
Western equine encephalitis
Dengue
Yellow fever
Rift Valley Fever
West Nile
Crimean Congo Hem. fever
Hantaan

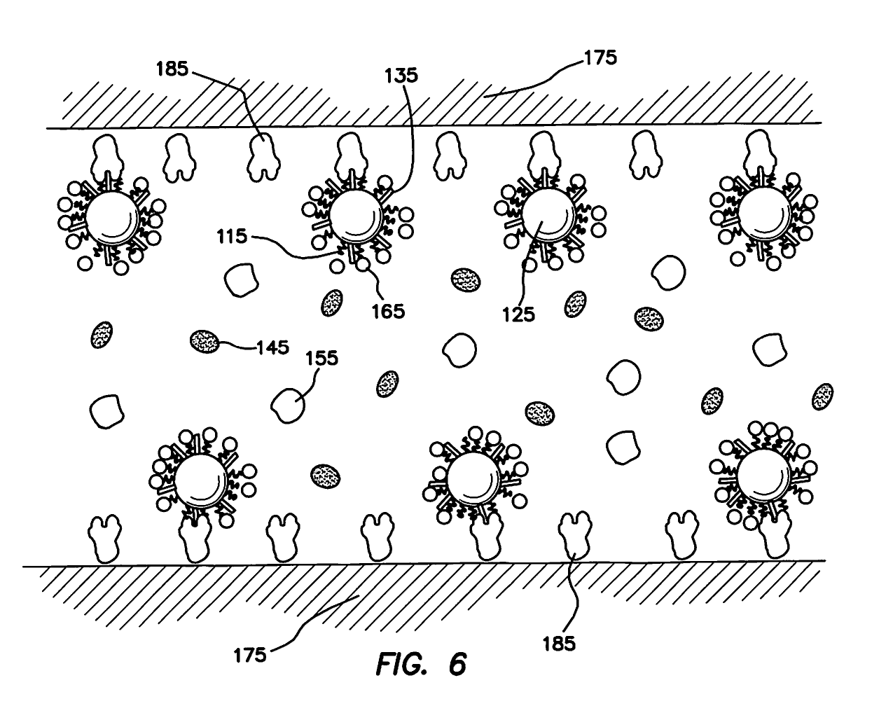

As shown in FIG. 6, removal of the microbubbles 125 is accomplished by passing the blood and contained microbubbles 125 of the subject over or through a substrate or membrane 175 which removes the microbubbles 125 from the blood while allowing other elements to pass unencumbered. In one embodiment, the substrate 175 comprises or includes Avidin and/or Streptavidin 185 which captures the microbubbles 125 using its strong affinity for the PEG-Biotin 135 formed with, or attached to, the microbubbles 125. Once filtered of the microbubbles 125, the blood is returned into the bloodstream of the subject. In this embodiment, the substrate 175 may be integrated into a machine akin to a dialysis machine. In an alternative embodiment, a subject's blood is selectively extracted from the subject after which the microbubbles 125 are added to the subject's blood outside of the subject. The blood, along with the added microbubbles 125, is then passed over or through the substrate 175 as described above. In this embodiment, the microbubbles never enter the subject's bloodstream. This described processes lower the subject's viral titer in the blood (i.e., the concentration of infectious viral particles per milliliter of suspension fluid).

In another embodiment, rather than using substrate 175, a centrifuge is used to separate or remove the microbubbles 125 and attached envelope viruses from the blood. In this embodiment, microbubbles 125 and attached envelope viruses 165 are separated from the core constituents forming the blood such that the microbubbles 125 and attached envelope viruses 165 can be separated or removed from the blood.

In one exemplary embodiment, 288 billion or more envelope viruses having a size of a HIV particle can be removed per ml of microbubbles 125 having 3 micron diameters. Microbubbles having a 3 micron diameter have a surface area of 28 micrometers². The average HIV particle has a surface area of 0.15 micrometers². With 50% of the surface area of the HIV particles attached to the microbubble 125 and 1 micrometer² left available for spacing, approximately 360 HIV particles can attach to a microbubble 125. Thus, a concentration of 1.6×10⁹ microbubbles per ml has the potential to sequester up to 576 billion enveloped virus particles. If the recovery rate is 50%, 288 billion enveloped virus particles can be sequestered. If the recovery rate is more or less than 50% the number of sequestered virus particles increases or decreases, respectively.

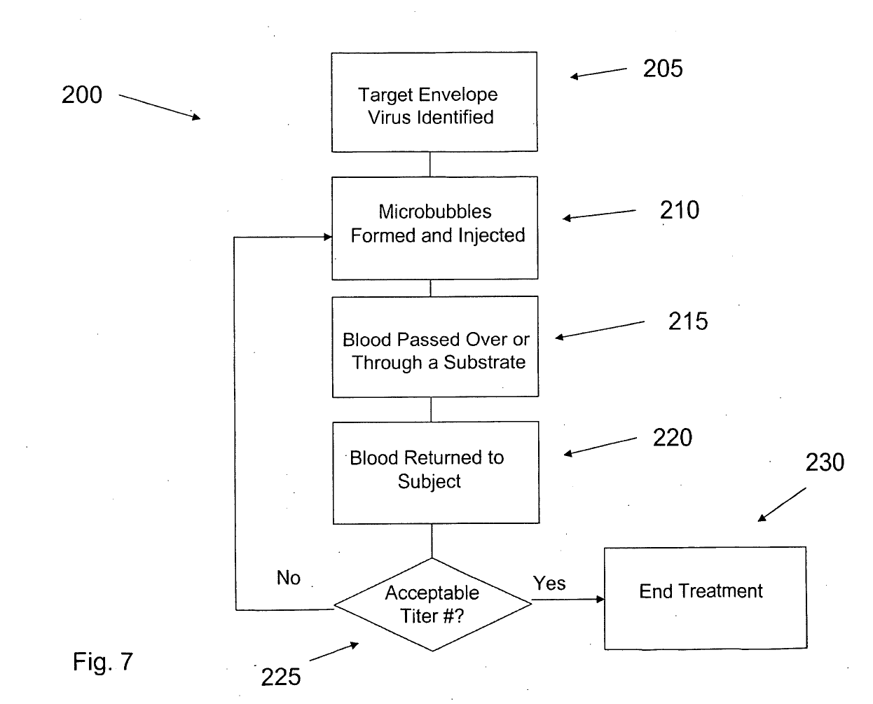

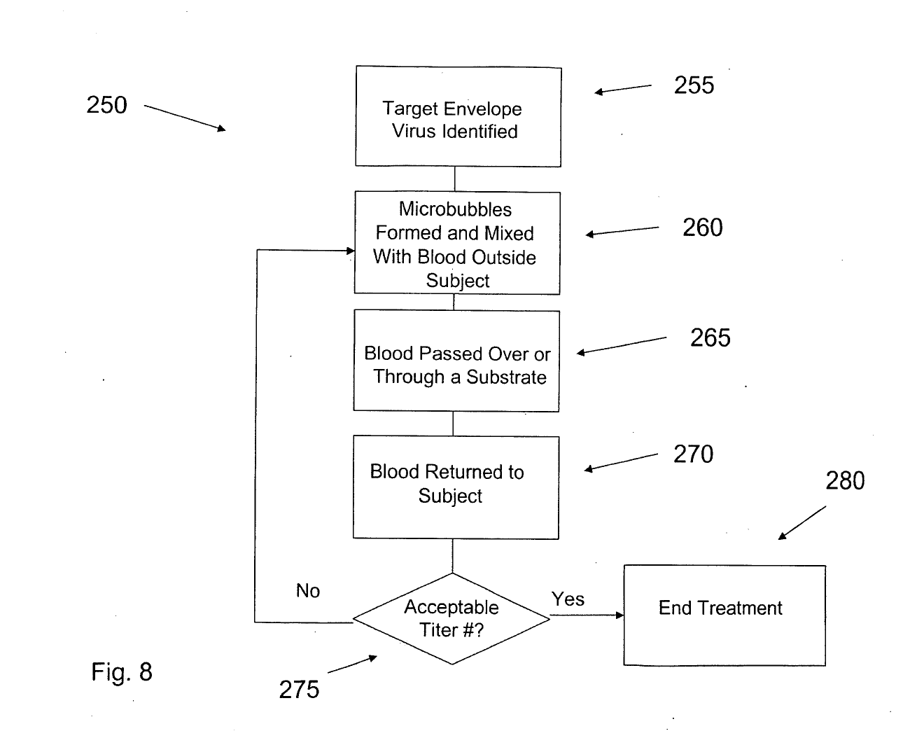

FIG. 7 shows a flow chart 200 of one exemplary method of utilizing the microbubbles 125 described herein. At 205, an envelope virus is identified in a human or animal subject. At 210, an appropriate volume of microbubbles 125 formed to target the identified virus using phosphorothioate DNA oligonucleotides 115 and/or phosphorothioate RNA oligonucleotides (or other polyanions) and PEG-Biotin 135 (or other conjugation systems) attached thereto is injected into said subject. At 215, after a pre-established time period has passed, the blood from the subject is passed over or through a Streptavidin substrate (or other substrate) removing the microbubbles 125 and attached envelope virus particles 165. At 220, the blood is returned to the subject. At 225, the subject's titer is measured to determine whether additional treatment is required. If not, at 230, the treatment ends. If so, the flow chart 200 loops back to 210. FIG. 8 shows an alternative flow chart 250 detailing a method whereby microbubbles 125 do not enter a subject's bloodstream. At 255, an envelope virus is identified in a human or animal subject. At 260, a subject's blood is selectively extracted and mixed outside of the subject's body with an appropriate volume of microbubbles 125 formed to target the identified virus using phosphorothioate DNA oligonucleotides 115 and/or phosphorothioate RNA oligonucleotides (or other polyanions) and PEG-Biotin 135 (or other conjugation systems) attached thereto. At 265, after a pre-established time period has passed, the blood and microbubble mixture is passed over or through a Streptavidin substrate (or other substrate) removing the microbubbles 125 and attached envelope virus particles 165. At 270, the blood is returned to the subject. At 275, the subject's titer is measured to determine whether additional treatment is required. If not, at 280, the treatment ends. If so, the flow chart 250 loops back to 260.

Those skilled in the art will recognize that liposomes may be used in place of, or in combination with, the microbubbles 125 described above. The liposomes are essentially microbubbles without the gas.

The embodiments of the present invention are used to lower a subject's viral titer thus inhibiting the reproductive and symptomatic progression of a virus. For example, decreasing the viral titer associated with influenza infection provides time for the subject's immune system to gain an advantage over the virus. In another example, decreasing the viral titer associated with Hepatitis C prolongs deterioration and other systems caused by the virus. In another example, decreasing the viral titer associated with HIV prolongs and/or prevents the onset of AIDS.

Although the invention has been described in detail with reference to several embodiments, additional variations and modifications exist within the scope and spirit of the invention as described and defined in the following claims.

We claim:

1. A microbubble comprising:
a gas;
a polyanion;
a conjugation system; and
one or more of the following:
a protein;
a lipid;
a cationic protein and
a cationic lipid.

2. The microbubble of claim 1 wherein said gas is selected from the group consisting of: hexafluoro acetone, isopropyl acetylene, allene, tetrafluoro-allene, boron trifluoride, isobutane, 1,2-butadiene, 2,3-butadiene, 1,3-butadiene, 1,2,3-trichloro-2-fluoro-1,3-butadiene, 2-methyl-1,3-butadiene, hexafluoro-1,3-butadiene, butadiyne, 1-fluoro-butane, 2-methyl-butane, decafluorobutane, 1-butene, 2-butene, 2-methyl-1-butene, 3-methyl-1-butene, perfluoro-1-butene, perfluoro-2-butene, 4-phenyl-3-butene-2-one, 2-methyl-1-butene-3-yne, butyl nitrate, 1-butyne, 2-butyne, 2-chloro-1,1,1,4,4,4-hexafluorobutyne, 3-methyl-1-butyne, perfluoro-2-butyne, 2-bromobutyraldehyde, carbonyl sulfide, crotononitrile, cyclobutane, methyl-cyclobutane, octafluoro-cyclobutane, perfluorocyclobutene, 3-chlorocyclopentene, octafluorocyclopentene, cyclopropane, 1,2-dimethyl-cyclopropane, 1,1-dimethylcyclopropane, 1,2-dimethyl-cyclopropane, ethylcyclopropane, methylcyclopropane, diacetylene, 3-ethyl-3-methyl diaziridine, 1,1,1-trifluorodiazoethane, dimethylamine, hexafluorodimethylamine, dimethylethylamine, bis(dimethylphosphine)amine, perfluorohexane, 2,3-dimethyl-2-norbornane, perfluorodimethylamine, dimethyloxonium chloride, 1,3-dioxolane-2-one, 4-methyl-1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1,1-dichloro-2-fluoroethane, 1-chloro-1,1,2,2-tetrafluoroethane, 2-chloro-1,1-difluoroethane, chloroethane, chloropentafluoroethane, dichlorotrifluoroethane, fluoroethane, hexafluoroethane, nitropentafluoroethane, nitrosopentafluoroethane, perfluoroethylamine, ethyl vinyl ether, 1,1-dichloroethane, 1,1-dichloro-1,2-difluoroethane, 1,2-difluoroethane, methane, trifluoromethanesulfonylchloride, trifluoromethanesulfonylfluoride, bromodifluoronitrosomethane, bromofluoromethane, bromochlorofluoromethane, bromotrifluoromethane, chlorodifluoronitromethane, chlorodinitromethane, chlorofluoromethane, chlorotrifluoromethane, chlorodifluoromethane, dibromodifluoromethane, dichlorodifluoromethane, dichlorofluoromethane, difluoromethane, difluoroiodomethane, disilanomethane, fluoromethane, iodomethane, iodotrifluoromethane, nitrotrifluoromethane, nitrosotrifluoromethane, tetrafluoromethane, trichlorofluoromethane, trifluoromethane, 2-methylbutane, methyl ether, methyl isopropyl ether, methyllactate, methylnitrite, methylsulfide, methyl vinyl ether, neon, neopentane, nitrogen (N.sub.2), nitrous oxide, 1,2,3-nonadecane-tricarboxylic acid-2-hydroxytrimethylester, 1-nonene-3-yne, oxygen (O.sub.2), 1,4-pentadiene, n-pentane, perfluoropentane, 4-amino-4-methylpentan-2-one, 1-pentene, 2-pentene(cis), 2-pentene(trans), 3-bromopent-1-ene, perfluoropent-1-ene, tetrachlorophthalic acid, 2,3,6-trimethylpiperidine, propane, 1,1,1,2,2,3-hexafluoropropane, 1,2-epoxypropane, 2,2-difluoropropane, 2-aminopropane, 2-chloropropane, heptafluoro-1-nitropropane, heptafluoro-1-nitrosopropane, perfluoropropane, propene, hexafluoropropane, 1,1,1,2,3,3-hexafluoro-2,3dichloropropane, 1-chloropropane, chloropropane-(trans), 2-chloropropane, 3-fluoropropane, propyne, 3,3,3-trifluoropropyne, 3-fluorostyrene, sulfur hexafluoride, sulfur (di)-decafluoride(S.sub.2F.sub.10), 2,4-diaminotoluene, trifluoroacetonitrile, trifluoromethyl peroxide, trifluoromethyl sulfide, tungsten hexafluoride, vinyl acetylene, vinyl ether, and xenon.

3. The microbubble of claim 1 wherein said polyanion is selected from the group consisting of: phosphorothioate 2′O-methyl DNA and RNA polycystosine oligonucleotides and degenerate phosphorothioate randomer oligonucleotides, polyoxometalates, polysulfonates, polyhydroxycarboxylates and sulfonated saccharides.

4. The microbubble of claim 1 wherein said conjugation system is selected from the group consisting of: Succinimidyl 6-hydrazinonicotinamide acetone hydrazone, Succinimidyl 4-formylbenzoate, all Hy Nic conjugation systems, all His-tag (polyhistidine) protein conjugation systems, streptavidin, avid in biotinylated polyethylene glycol, biotinylated proteins, biotinylated olinucleotides.

5. A liposome comprising:
a polyanion;
a conjugation system; and
one or more of the following:
a protein;
a lipid;
a cationic protein and
a cationic lipid.

6. The liposome of claim 5 wherein said gas is selected from the group consisting of: hexafluoro acetone, isopropyl acetylene, allene, tetrafluoro-allene, boron trifluoride, isobutane, 1,2-butadiene, 2,3-butadiene, 1,3-butadiene, 1,2,3-trichloro-2-fluoro-1,3-butadiene, 2-methyl-1,3-butadiene, hexafluoro-1,3-butadiene, butadiyne, 1-fluoro-butane, 2-methyl-butane, decafluorobutane, 1-butene, 2-butene, 2-methyl-1-butene, 3-methyl-1-butene, perfluoro-1-butene, perfluoro-2-butene, 4-phenyl-3-butene-2-one, 2-methyl-1-butene-3-yne, butyl nitrate, 1-butyne, 2-butyne, 2-chloro-1,1,1,4,4,4-hexafluorobutyne, 3-methyl-1-butyne, perfluoro-2-butyne, 2-bromobutyraldehyde, carbonyl sulfide, crotononitrile, cyclobutane, methyl-cyclobutane, octafluoro-cyclobutane, perfluorocyclobutene, 3-chlorocyclopentene, octafluorocyclopentene, cyclopropane, 1,2-dimethyl-cyclopropane, 1,1-dimethylcyclopropane, 1,2-dimethyl-cyclopropane, ethylcyclopropane, methylcyclopropane, diacetylene, 3-ethyl-3-methyl diaziridine, 1,1,1-trifluorodiazoethane, dimethylamine, hexafluorodimethylamine, dimethylethylamine, bis(dimethylphosphine)amine, perfluorohexane, 2,3-dimethyl-2-norbornane, perfluorodimethylamine, dimethyloxonium chloride, 1,3-dioxolane-2-one, 4-methyl-1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1,1-dichloro-2-fluoroethane, 1-chloro-1,1,2,2-tetrafluoroethane, 2-chloro-1,1-difluoroethane, chloroethane, chloropentafluoroethane, dichlorotrifluoroethane, fluoroethane, hexafluoroethane, nitropentafluoroethane, nitrosopentafluoroethane, perfluoroethylamine, ethyl vinyl ether, 1,1-dichloroethane, 1,1-dichloro-1,2-difluoroethane, 1,2-difluoroethane, methane, trifluoromethanesulfonylchloride, trifluoromethanesulfonylfluoride, bromodifluoronitrosomethane, bromofluoromethane, bromochlorofluoromethane, bromotrifluoromethane, chlorodifluoronitromethane, chlorodinitromethane, chlorofluoromethane, chlorotrifluoromethane, chlorodifluoromethane, dibromodifluoromethane, dichlorodifluoromethane, dichlorofluoromethane, difluoromethane, difluoroiodomethane, disilanomethane, fluoromethane, iodomethane, iodotrifluoromethane, nitrotrifluoromethane, nitrosotrifluoromethane, tetrafluoromethane, trichlorofluoromethane, trifluoromethane, 2-methylbutane, methyl ether, methyl isopropyl ether, methyllactate, methylnitrite, methylsulfide, methyl vinyl ether, neon, neopentane, nitrogen (N.sub.2), nitrous oxide, 1,2,3-nonadecane-tricarboxylic acid-2-hydroxytrimethylester, 1-nonene-3-yne, oxygen (O.sub.2), 1,4-pentadiene, n-pentane, perfluoropentane, 4-amino-4-methylpentan-2-one, 1-pentene, 2-pentene(cis), 2-pentene(trans), 3-bromopent-1-ene, perfluoropent-1-ene, tetrachlorophthalic acid, 2,3,6-trimethylpiperidine, propane, 1,1,1,2,2,3-hexafluoropropane, 1,2-epoxypropane, 2,2-difluoropropane, 2-aminopropane, 2-chloropropane, heptafluoro-1-nitropropane, heptafluoro-1-nitrosopropane, perfluoropropane, propene, hexafluoropropane, 1,1,1,2,3,3-hexafluoro-2,3dichloropropane, 1-chloropropane, chloropropane-(trans), 2-chloropropane, 3-fluoropropane, propyne, 3,3,3-trifluoropropyne, 3-fluorostyrene, sulfur hexafluoride, sulfur (di)-decafluoride(S.sub.2 F.sub. 10), 2,4-diaminotoluene, trifluoroacetonitrile, trifluoromethyl peroxide, trifluoromethyl sulfide, tungsten hexafluoride, vinyl acetylene, vinyl ether, and xenon.

7. The liposome of claim 5 wherein said polyanion is selected from the group consisting of: phosphorothioate 2′O-methyl DNA and RNA polycystosine oligonucleotides and degenerate phosphorothioate randomer oligonucleotides, polyoxometalates, polysulfonates, polyhydroxycarboxylates and sulfonated saccharides.

8. The liposome of claim 5 wherein said conjugation system is selected from the group consisting of: Succinimidyl 6-hydrazinonicotinamide acetone hydrazone, Succinimidyl 4-formylbenzoate, all Hy Nic conjugation systems, all His-tag (polyhistidine) protein conjugation systems, streptavidin, avidin biotinylated polyethylene glycol, biotinylated proteins, biotinylated olinucleotides.

9. A method for lowering a viral titer comprising:
injecting a subject with microbubbles comprising:
a gas;
a polyanion;
a conjugation system; and
one or more of the following:
a protein;
a lipid;
a cationic protein and
a cationic lipid;
allowing said microbubbles to remain in said subject for a pre-established time period; and
once said pre-established time period expires, removing said microbubbles and attached virus particles from said blood.

10. The method of claim 9 further comprising passing said blood over or through a substrate having one or more conjugation systems to remove said microbubbles and attached virus particles from said blood.

11. The method of claim 9 further comprising placing said blood in a centrifuge to separate for removal said microbubbles and attached virus particles from said blood.

12. A method for lowering a viral titer comprising:
removing blood of a subject;
combining said removed blood of a subject with a solution of microbubbles, wherein said microbubbles comprise:
a gas;
a polyanion;
a conjugation system; and
one or more of the following:
a protein;
a lipid;
a cationic protein and
a cationic lipid;
allowing said microbubbles and blood to interact for a pre-established time period;
once said pre-established time period expires, removing said microbubbles and attached virus particles from said blood.

13. The method of claim 12 further comprising passing said blood over or through a substrate having one or more conjugation systems to remove said microbubbles and attached virus particles from said blood.

14. The method of claim 12 further comprising placing said blood in a centrifuge to separate for removal said microbubbles and attached virus particles from said blood.

15. A system for lowering a viral titer comprising:
a plurality of microbubbles configured to attract virus particles in blood, wherein said microbubbles comprise:
a gas;
a polyanion;
a conjugation system; and
one or more of the following:
a protein;
a lipid;
a cationic protein and
a cationic lipid; and
means for removing microbubbles and attached virus particles from said blood.

16. The system of claim 15 wherein said means for removing microbubbles and attached virus particles from said blood comprises a substrate having one or more conjugation systems.

17. The system of claim 15 wherein said means for removing microbubbles and attached virus particles from said blood comprises a centrifuge.

18. A method for lowering a viral titer comprising:
injecting a subject with liposomes comprising:
a polyanion;
a conjugation system; and
one or more of the following:
a protein;
a lipid;
a cationic protein and
a cationic lipid;
allowing said liposomes to remain in said subject for a pre-established time period; and
once said pre-established time period expires, removing said liposomes and attached virus particles from said blood.

19. The method of claim 18 further comprising passing said blood over or through a substrate having one or more conjugation systems to remove said liposomes and attached virus particles from said blood.

20. The method of claim 18 further comprising placing said blood in a centrifuge to separate for removal said liposomes and attached virus particles from said blood.

21. A method for lowering a viral titer comprising:
removing blood of a subject;
combining said removed blood of a subject with a solution of liposomes, wherein said liposomes comprise:
a polyanion;
a conjugation system; and
one or more of the following:
a protein;
a lipid;
a cationic protein and
a cationic lipid;
allowing said liposomes and blood to interact for a pre-established time period;
once said pre-established time period expires, removing said liposomes and attached virus particles from said blood.

22. The method of claim 21 further comprising passing said blood over or through a substrate having one or more conjugation systems to remove said liposomes and attached virus particles from said blood.

23. The method of claim 21 further comprising placing said blood in a centrifuge to separate for removal said liposomes and attached virus particles from said blood.

24. A system for lowering a viral titer comprising:
a plurality of liposomes configured to attract virus particles in blood, wherein said liposomes comprise:
a gas;
a polyanion;
a conjugation system; and
one or more of the following:
a protein;
a lipid;
a cationic protein and
a cationic lipid; and
means for removing liposomes and attached virus particles from said blood.

25. The system of claim 24 wherein said means for removing liposomes and attached virus particles from said blood comprises a substrate having one or more conjugation systems.

26. The system of claim 24 wherein said means for removing liposomes and attached virus particles from said blood comprises a centrifuge.
